(19) United States
(12) Patent Application Publication
(10) Pub. No.: US 2007/0161104 A1
(43) Pub. Date: Jul. 12, 2007

(54) Waste management facilities for the production of hydrogen gas and reduction of greenhouse gases

(76) Inventor: Douglas Diemer, Fountain Hills, AZ (US)

Correspondence Address: WELSH & FLAXMAN LLC, 2000 DUKE STREET, SUITE 100, ALEXANDRIA, VA, 22314, US

(21) Appl. No.: 11/652,135

(22) Filed: Jan. 11, 2007

Related U.S. Application Data
(60) Provisional application No. 60/757,873, filed on Jan. 11, 2006.
(60) Provisional application No. 60/761,371, filed on Jan. 24, 2006.
(60) Provisional application No. 60/854,436, filed on Oct. 26, 2006.

(51) Int. Cl. C12M 3/00

(52) U.S. Cl. 435/289.1

(57) ABSTRACT

A waste management facility is provided with operational control of a waste mass for optimum hydrogen production. The facility includes an air circulation control facility cooperating with the waste mass, a liquid introduction system cooperating within the waste mass, a monitoring mechanism for optimum operation of the waste management facility, a control mechanism for adjusting air circulation and liquid introduction thereby controlling waste mass temperature and pH to create an environment to deactivate methanogenic bacteria and isolate and maximize the number of hydrogen producing bacteria and a gas collection system for collecting the hydrogen produced by the hydrogen producing bacteria.

### CROSS REFERENCE TO RELATED APPLICATIONS

This application claims the benefit of U.S. Provisional Application Ser. Nos. 60/757,873, entitled “THE PROCESS FOR DESIGN, OPERATION AND/OR TREATMENT OF SOLID WASTE AND SOLID WASTE MANAGEMENT FACILITIES FOR THE PRODUCTION OF HYDROGEN GAS”, filed Jan. 11, 2006, 60/761,371, entitled “THE PROCESS FOR DESIGN, OPERATION AND/OR TREATMENT OF SOLID WASTE AND SOLID WASTE MANAGEMENT FACILITIES FOR THE PRODUCTION OF HYDROGEN GAS”, filed Jan. 24, 2006, and 60/854,436, entitled “THE PROCESS FOR DESIGN, OPERATION AND/OR TREATMENT OF SOLID WASTE AND SOLID WASTE MANAGEMENT FACILITIES FOR THE PRODUCTION OF HYDROGEN GAS”, filed Oct. 26, 2006.

### BACKGROUND OF THE INVENTION

1. Field of the Invention

The present invention is directed to the design of containment and control facilities, the treatment and management of solid and liquid wastes, and the operating techniques that collectively or individually provide for aerobic and/or anaerobic biodegradation of solid, semi-solid, and liquid wastes. This is achieved by the developing and/or maintaining of specific waste mass temperatures that preclude methanogenic activity, establishing and/or maintaining a specific waste mass moisture content, maintaining specific waste mass pH range/levels, and establishing and maintaining micro-nutrient and/or trace element conditions within the waste mass that are conducive to the growth, sustainability, and maximization of hydrogen gas yield by hydrogen producing bacteria and/or spores. These steps are utilized in the production of hydrogen gas for commercial, industrial, or beneficial use and for the reduction or elimination of methane gas emissions.

2. Description of the Prior Art

The landfilling, or burying, of municipal solid waste has been utilized for decades. In the United States and in many developed countries, federal, state, and local regulations mandate allowable operating techniques, and environmental protection systems, at current state-of-the-art landfills. Recycling of certain solid and liquid wastes is also becoming more popular and is legislated in certain states. Recycling techniques vary depending on material type and include recent developments in technology for composting of municipal solid waste.

More recent conventional landfills (including closed landfills) are typically designed and operated to restrict moisture infiltration, and to place and keep waste as dry as possible throughout the operating life of the facility and through closure and post-closure maintenance periods. These design and operating conditions are employed to reduce the potential for leaching of contaminants from the waste mass, potentially impacting groundwater and/or surface waters with contaminated water referred to as “leachate.” Though more modern, recently constructed landfills are typically lined with natural and/or synthetic liners to contain leachate, in general, the goal of operations is to maintain dry conditions to preclude the formation of leachate and impacts to surface and groundwater. With the exception of leachate collection and control systems, and sometimes landfill gas collection systems for the extraction and reuse of methane, the typical approach to more recent landfill waste mass treatment or operations is generally passive with attendant monitoring.

While current landfill operations and management techniques typically are focused on preventing moisture infiltration into the waste, landfills in wetter climates will always be more susceptible to the formation of landfill gas. For landfill gas generated within the typical landfill today, the primary goal of collection of this gas has been either for air-pollution control and/or for the beneficial reuse of the methane contained within the gas. For controlling air pollution, landfill gas that may otherwise be passively vented from the landfill to the surrounding air is collected and controlled. This gas, which typically contains a relatively large percentage of methane (typically approximately 45-50% methane, 45-50% carbon dioxide, with minor concentrations of other gases), is commonly incinerated within an enclosed flare for destroying gases otherwise considered to be air pollutants.

Other landfills, especially larger landfills in wetter climates, generate abundant landfill gas. It is the goal of these landfills to collect the methane-laden gas, and to use the methane contained within the gas as a fuel for producing other products or energy. Standard landfill design and operations however, typically do not preclude the escape of methane gas and other landfill gases through the surface of the landfill to the atmosphere. Both methane and carbon dioxide are greenhouse gases that may contribute to global warming. Methane is estimated to have as much as 20-23 times the long-term greenhouse gas and global warming effects as carbon dioxide.

Typical landfill operations of today sometimes utilize the methane gas component of the landfill gas as fuel for internal combustion or turbine engines to generate electricity. Though this is a very viable, productive use of the methane component of typical landfill gas, burning of this fuel can result in emissions of additional air pollutants when using today's engine technology. Some facilities and research are focused on the conversion of methane gas collected within the current state-of-the-art landfill to free hydrogen gas using various conversion technologies. These methods are recognized as relatively capital intensive, relatively inefficient, and energy/cost intensive.

A very recent development is called “bioreactor landfills” and seeks to create specific moisture and temperature levels conducive to the rapid, anaerobic, methanogenic degradation of solid wastes. The bioreactor landfill operating criteria generally require the placement and/or injection of moisture/water or liquid waste into the landfill, and the maintenance of specific moisture conditions, anaerobic conditions, and relatively lower internal landfill temperatures, to promote optimum conditions for methanogenic bacteria and the rapid degeneration of wastes. The rapid degradation has the benefit of earlier waste stabilization and faster waste degradation allowing more waste to be placed in the same area/volume when compared to past operating techniques, providing for a greater net landfill capacity. Bioreactor landfills also typically generate a higher volume of reusable methane-containing landfill gas, but also may release substantially more methane and carbon dioxide (greenhouse gases) to the atmosphere through the surface of the landfill when compared to a standard landfill.

Historical and current landfill design and operation technologies attempt to achieve and maintain anaerobic conditions throughout the waste mass by the placement of appropriate daily and intermediate cover on the waste mass side-slope, top, and inactive exposed areas. Generally, the cover utilized, especially for inactive areas of the landfill, consists of a minimum of 12-inches of earthen/soil materials. Soils are used to secure the recently inactive outer boundaries of the waste mass against air intrusion and landfill gas migration from the waste mass (and for other purposes, including but not limited to erosion control, vehicle and equipment accessibility, odor control, etc). With respect to gas generation and extraction for beneficial use of the gas, one of the primary purposes of consistent, relatively thick, impervious soil cover is to create a boundary that prevents air intrusion into the landfill to minimize or eliminate the potential for aerobic conditions to develop, and to create a temperature and oxygen (lack of) environment conducive to the formation of methanogenic bacteria. Methanogenic activity is the production of methane and carbon dioxide through the fermentation of simple organic compounds or oxidation of H₂ under anaerobic conditions. Synthetic materials are sometimes used to provide the cover seal on exterior landfill surfaces, where appropriate soil materials may not be available, for those purposes indicated above as for soil cover. Where this general operating technique is employed and where appropriate, methane gas is typically targeted as a fuel for use in electrical generation systems or for other purposes related to beneficial reuse.

For anaerobic methanogenic bacteria to survive and to thrive, certain temperature and pH conditions are required. Anaerobic methanogenic bacteria survive and are excellent methane producers at temperatures below approximately 150° Fahrenheit. Further, methanogenic bacteria are most active and thrive in a relatively neutral acidic environment, in a pH range of approximately 6.5 to 7.5.

For current landfill design and operating technology, air intrusion and the development of higher internal waste mass temperatures (higher than approximately 130° Fahrenheit) is generally deliberately avoided, in order to reduce the potential for generating temperatures conducive to spontaneous combustion (internal waste mass combustion, or landfill fire) and to maintain a stable anaerobic environment and temperatures conducive to the formation and sustainability of methanogenic bacteria.

With the continuing depletion of available landfill resources, evolving interest in renewable/recyclable materials, and the fact landfills themselves can be a long-term environmental liability concern, alternatives have recently been developed for the processing and reuse of solid wastes. These facilities typically employ closed systems/vessels that rely on biological processes and creating optimum conditions within the vessel to accelerate waste decomposition, provide for volume reduction, and produce a re-useable product. Air and/or water are added, typically along with other nutrients, to achieve conditions that are the most favorable for rapid bioconversion. These mechanical, biological treatment processes are used to more rapidly convert the organic fraction of waste into usable end products such as compost or bio-fuel. Inorganic materials, such as plastics and metals are removed from the waste mass before or during the process and recycled. Though these mechanical, biological systems typically yield a homogeneous organic material preferred for the process described herein for producing hydrogen gas, their use has been to generate organic compost or bio-fuel (for incineration energy). The closed vessel can be used to manage solid wastes using the methods prescribed herein, with optimum control capability, for producing hydrogen gas.

As those skilled in the art will certainly appreciate, U.S. Pat. No. 6,860,996 to Nioke et al., which is incorporated herein by reference, teaches the chemistry involved with producing hydrogen from organic waste by mechanically heating and controlling pH so that methane producing bacteria are decreased.

In the patent literature there are several references to municipal solid and liquid waste management, solid waste landfill design and operating techniques, management equipment and containment facilities for the management of solid and liquid waste, for the mechanical processing of solid and liquid waste for landfill volume reduction, recycling, and/or the production of reusable materials, and specifically for the collection and reuse of methane gas.

U.S. Pat. No. 6,283,676, issued Sep. 4, 2001, describes a method of placing waste and horizontal piping layers for the introduction/re-introduction of liquids to accelerate anaerobic and/or aerobic degradation of municipal solid waste to increase landfill capacity. The object of the invention is to provide a method for degrading solid waste that increases landfill density and capacity by accelerated landfill aerobic and/or anaerobic degradation, provides a method for degrading solid waste that improves degradation by-product quality, and for reducing noxious landfill gas emissions.

U.S. Pat. No. 5,984,580, issued Nov. 16, 1999, describes a method of producing a landfill wherein the waste to be landfilled is comminuted to yield homogeneously-sized waste particles, mixed with liquid wastes such as water treatment sludges and the like, and then placed into a sanitary containment site with a leachate and gas collection system such that there is a uniform moisture distribution throughout the waste mass. Leachate reintroduced into the waste mass is uniformly re-introduced into the waste mass so as to maintain the uniform moisture distribution. The landfill produced thereby maximizes anaerobic fermentation within the waste mass, thereby maximizing the production of valuable landfill gases.

U.S. Pat. No. 6,481,929, issued Nov. 19, 2002, describes a process for converting municipal solid waste landfills to aerobic conditions that will allow for a highly accelerated and enhanced bioreduction of landfill mass, followed by the optional excavation of the landfill cell materials subsequent to the bioreduction process, separation of excavated materials using trommels, screens, and other devices as necessary, production of useable compost materials, and reclamation of recyclable plastics, metal, and glass.

U.S. Pat. No. 6,945,484, issued Sep. 20, 2005 discloses a method for processing municipal waste substantially that reduces the volume of material to be disposed into a landfill by increasing the volume of materials of value collected from the municipal waste and by composting the remaining materials to create a salable product. The process includes the removal of materials of value by magnetic devices to extract ferrous materials, by an air classifier to extract light plastic materials, by an eddy current mechanism to extract aluminum materials, and by a plastic sort mechanism to extract large plastic materials. Composting can be accomplished with a composting vessel that is located in the process either before the materials of value are removed or afterward. A grinding of the materials before a final screening allows the commercial grade compost to be separated from the materials that are to be sent to the landfill for final disposal. An alternative embodiment of the process utilizes a steam pressure vessel to disintegrate the waste material and to destroy any pathogens before materials of value are removed and the small and fine materials are composted. Large materials can be gasified to create heat energy for use in the steam pressure vessel.

U.S. Pat. No. 4,369,402, issued Aug. 2, 1983, describes a multi-step approach for accelerating gas production by bioleaching organic materials, including 1) inoculation with anaerobic, acid-forming microorganisms, 2) acid-phase digestion of substrates, and 3) methane-phase digestion.

U.S. Pat. No. 4,643,111, issued Feb. 17, 1987, describes a landfill completely enclosed within a large vapor-tight container. Methane formed by anaerobic fermentation within the landfill migrates to the top of the enclosure, where it is collected.

U.S. Pat. No. 4,798,801, issued Jan. 17, 1989, describes a means for generating methane by anaerobic fermentation of waste. Generally describes a process of developing a series of operating solid waste cells, utilizing CO₂ gases generated and then extracted/concentrated from the landfill gas from a neighbor cell for injecting into neighboring cells for forcing out remaining oxygen, thereby promoting anaerobic fermentation by methanogenic bacteria for the production and collection of methane.

U.S. Pat. No. 5,139,365, issued Aug. 18, 1992, describes a method for injecting wastes, primarily liquid or mixed liquid wastes, into an existing landfill to more effectively utilize landfill airspace, and to develop a controlled landfill conducive to the growth of methanogenic organisms.

### SUMMARY OF THE INVENTION

It is, therefore, an object of the present invention to provide a waste management facility provided with operational control of a waste mass for optimum hydrogen production. The facility includes an air circulation control facility cooperating with the waste mass, a liquid introduction system cooperating within the waste mass, monitoring means for monitoring at least temperature, waste mass moisture content and waste mass pH, control means for adjusting air circulation and liquid introduction thereby controlling waste mass temperature and pH to create an optimum environment to deactivate methanogenic bacteria and isolate and maximize the number of hydrogen producing bacteria, and a gas collection system for collecting the hydrogen produced by the hydrogen producing bacteria

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic of a landfill facility in accordance with the present invention.

FIG. 2 is a detailed view of the Section II shown in FIG. 1.

FIG. 3 is a cross sectional schematic of a landfill facility in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

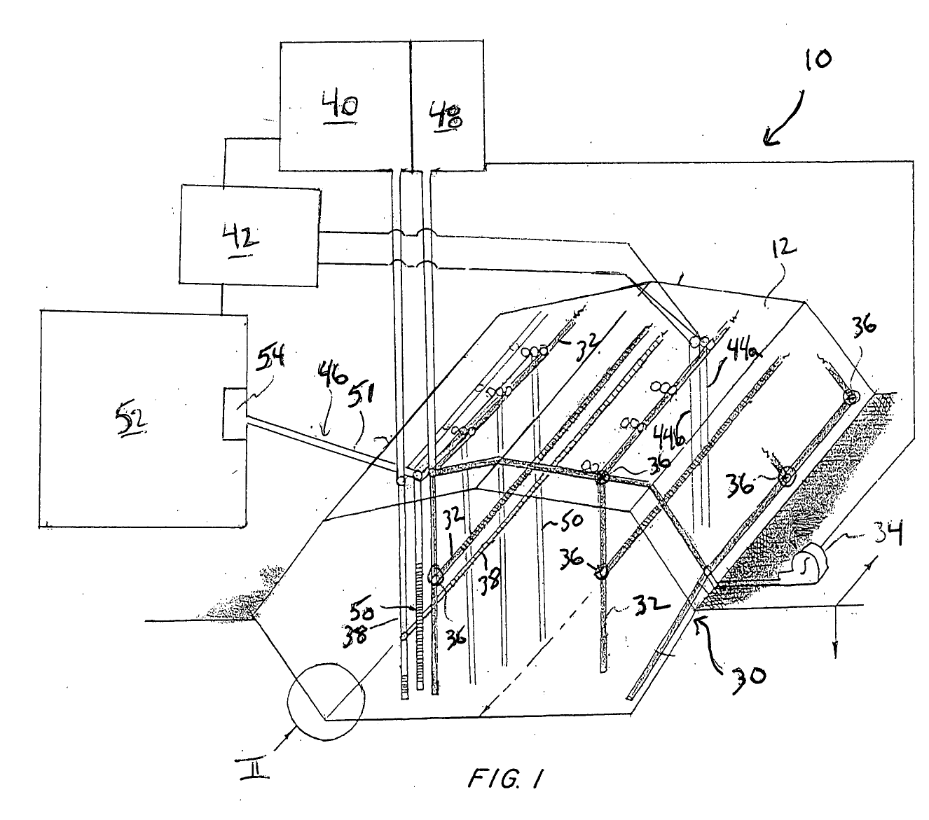

In accordance with the present invention, and with reference to FIGS. 1 to 3, a method is disclosed for the design, operation, and/or treatment of solid and liquid waste, as well as a solid and liquid waste management facility (for example, a landfill facility or vessel) that promotes an internal waste mass environment conducive to the activation, isolation, cultivation, and sustainability of anaerobic hydrogen producing bacteria and/or spores. The present method is applicable to new solid and/or liquid waste landfill development. The present method may also be applied in the conversion or enhancement of an existing active or closed solid waste landfill, for closed vessel applications, or for any activity relating to implementing the features and/or management and operational processes described herein for the transformation of typical solid and liquid wastes to hydrogen gas. Although not an exhaustive list, these activities may include (a) managing solid, semi-solid, and liquid wastes in order to create a relatively homogeneous internal waste mass condition; (b) incorporating specific alternative organic and/or inorganic materials into the waste mass at the appropriate mix/concentration; (c) placing specific bacteria-containing wastes or materials into the waste mass, including natural soils, or inoculating wastes with specific bacteria or specific bacteria containing materials; and, (d) precisely controlling internal waste mass temperatures by regulating and managing the waste mass environment to promote an internal waste mass temperature environment conducive to the activation, isolation and sustainability of hydrogen producing bacteria and/or spores.

The present invention achieves this by developing desired internal waste mass temperatures and specific biological spore-forming populations by short-term, or interim, controlled, aerobic heat-shock methods as discussed below in greater detail. The aerobic heat-shock methods are employed as pretreatment of the waste mass and specific inoculants, and are followed by anaerobic waste mass operations and/or combined aerobic/anaerobic operations.

The goals of the present invention are also achieved by maintaining a relative moisture condition within the waste mass that is conducive to the activation, isolation and sustainability of hydrogen producing bacteria and/or spores. This is achieved by various mechanism, including but not limited to, water injection through water supply piping or gas collection facilities, leachate recirculation through appropriately spaced vertical or horizontal water supply piping or gas collection facilities, and/or spraying or mixing the waste mass with water, leachate, and/or liquid wastes prior to placement within a landfill, closed vessel or other waste mass management unit.

In addition, the present method is implemented by maintaining a specific acidic environment within the waste mass by the uniform reintroduction of certain liquids, leachate, or gas condensate, as necessary, through various supply and distribution piping, and/or introducing directly as the waste is placed. The liquids or materials are introduced to produce or maintain an acidic environment, which is optimum for the activation, isolation and/or sustainability of hydrogen producing bacteria and/or spores for maximum hydrogen gas production.

In addition, the concepts of the present invention are implemented by placing certain wastes, soils, or specific other materials containing iron, phosphorous, nickel, nitrogen, etc. within the landfill waste mass. The purpose of this placement is the maintenance of an acidic environment directed toward bacterial energy metabolism based on volatile fatty acids. This placement also provides for the sustainability of a micronutrient/trace element environment concentration conducive to the activation, isolation and sustainability of hydrogen producing bacteria and/or spores for maximum hydrogen production.

The present invention also provides for the operation and monitoring of the landfill, closed vessel, or waste mass by the installed gas collection/control system to achieve and maintain internal waste mass partial gas pressures necessary for maximizing hydrogen gas production.

Any, or all of the above techniques may be applied to a new landfill development, to an existing landfill, closed landfill, closed vessel, or any waste mass management unit, for the conversion of that waste mass from its current state, to a condition that yields hydrogen gas.

As will be discussed below in greater detail, the present invention also includes the collection of gas generated from within a waste mass by an integrated gas collection system. This achieved by vertical and/or horizontal gas wells, lateral collection piping, and header/main delivery piping.

Closed vessel gas extraction equipment comprised of any methods or equipment for collecting the gas from within/generated within the waste mass may also be employed in the collection of gas without departing from the spirit of the present invention. Internal waste mass partial gas pressure and gas well monitoring equipment for determining waste mass partial pressure for maximizing hydrogen gas production and for determining appropriate extraction rates may also be implemented within the spirit of the present invention.

Transport piping for delivery of the collected gas to a gas processing and clean-up system facility for extraction of free hydrogen, by any existing, known, or otherwise developed extraction process is also used in accordance with the present invention for the developed free hydrogen gas or for liquefied gas that may be beneficially reused. The developed gas may also be applied for hydrogen fuel cell supply for commercial, industrial or other beneficial uses, and/or the use of free hydrogen gas for any other commercial, industrial, or beneficial reuse. The free hydrogen gas or liquefied gas may be temporarily stored for later use in energy production or fuel cells, and/or for future transport to certain other facilities for energy production, fuel cell supply, or other commercial, industrial or beneficial use.

A first expression of the invention is concentrated on the method of the design and management of solid waste and environmental control equipment and systems to yield a waste mass environment conducive to the activation, sustainability and efficiency of hydrogen producing bacteria and/or spores for the production of hydrogen gas for commercial, industrial, or other beneficial use. The method comprises designing, constructing, and/or operating, or otherwise converting or retrofitting an existing landfill unit, with appropriate features and/or equipment to attain an internal landfill, waste mass, or vessel condition specific to the activation, isolation and sustainability of hydrogen producing bacteria and/or spores.

A second expression of the invention is concentrated on the homogeneous placement of solid wastes, liquid wastes, and specific inoculating environment-sustaining materials or liquids within a waste management unit or vessel, or the conversion/retrofit of an existing waste mass, to create such conditions conducive to the activation, sustainability, and hydrogen producing efficiency of hydrogen producing bacteria and/or spores. This expression of the invention is drawn to the waste placement, treatment, or management method that produces the most homogeneous, consistent, internal landfill or waste mass condition, including waste type, waste moisture content, pH, trace element concentrations, and bacteria concentrations, for optimum waste mass control and management, and optimum free hydrogen gas production. The solid and liquid wastes, inoculating soil materials, other inoculants, catalyst trace elements, and pH control materials as needed, are placed uniformly and homogenously within the landfill or waste management vessel, to produce an environment conducive to the effective management of the entire waste mass, whereby as much of the waste mass as possible is maintained in the desired condition for optimum hydrogen production.

A third expression of the invention is concentrated on the incorporation, placement, or addition of specific (landfill cover) materials at landfills or within closed vessels, at the proper concentrations and at specific mix ratios, as hydrogen producing bacteria inoculants, for the introduction of catalyst trace elements, or as a substrate. This expression is drawn to the method of uniformly incorporating into the waste mass certain organic waste materials, including but not limited to municipal sewage sludge, or any other organic materials or liquid wastes or mixtures. This expression is also drawn to incorporating natural soils or soils mixed with any organic solid or liquid materials as listed above or otherwise available, for the purpose of developing a uniform, homogeneous solid waste mass, replete with naturally occurring hydrogen producing bacteria and/or spores for the later activation and cultivation of those bacteria for producing and sustaining an optimum environment conducive to the production of hydrogen gas. This expression is also drawn to the placement/introduction of soils, or other specific additives containing desired trace elements, including primarily (but not limited to) naturally occurring iron into the landfill/solid waste mass (or used as daily waste cover in a landfill), for providing desired conditions for later hydrogen production by developing and sustaining a waste mass environment replete with necessary trace elements for hydrogen producing anaerobic bacteria (condition that promotes sustainable iron-hydrogenase catalysis formation (or FeNi, etc.)). Typical desired waste placement, or waste mass thicknesses, prior to the placement or incorporation of certain organic, bacteria containing waste, or soil materials may vary depending on waste(s), soils, or liquids availability, but are placed as evenly and regularly as practical for the specific waste management facility to develop a total waste mass condition that is as homogeneous as practical given the timing of waste-stream receipts and availability.

This expression is further drawn to describe a method of waste and cover placement to avoid the incorporation of internal soil or other internal waste mass barriers that would contribute to a lesser homogeneous mass than otherwise obtained. This homogeneous waste mass contributes to the most effective application of external and internal waste mass design and control features, and results in optimum/higher levels of hydrogen gas production. This expression is further drawn to the development of similar conditions in a mechanical, closed vessel process, whereby the loading of organic wastes, liquid wastes, inoculants concentration, trace elements, and total waste mass condition is developed that is as homogeneous as practical for the efficient, optimum bacterial degradation of the waste mass.

The fourth expression of the invention is concentrated on the design and operating techniques utilized to control temperature within the waste mass. The purpose of temperature control facilities and management techniques is to develop relatively higher temperatures, versus conventional landfill or mechanical waste mass compost operating techniques. These higher temperatures are conducive to the inactivation of methanogenic bacteria and the activation, isolation, cultivation, and sustainability of hydrogen producing bacteria and/or spores. The prescribed means of elevating internal landfill, waste mass, or vessel temperatures is through the temporary and/or intermittent introduction of air to the waste mass to temporarily produce aerobic conditions and to temporarily achieve higher waste mass temperatures (in excess of approximately 165° Fahrenheit). These temperatures which are achieved by temporary aerobic operations of the waste mass, are required to inactivate methanogenic bacteria and to isolate hydrogen producing bacteria and/or sporulation.

The prescribed operating facilities for controlling temperature may include, but would not be limited to, the temporary introduction of air to the landfill, waste mass, or vessel, through various design features including vent piping or forced air systems, or other internal landfill or environmental control features. In closed vessels, venting or forced air can be used, in addition to other temperature control methods including insulation, heat exchangers, heating elements, water baths, and steam injection. Once the higher temperatures are achieved through aerobic operating techniques that are sufficient to inactivate methanogenic bacteria, design features and operating techniques are utilized to return the waste mass to anaerobic conditions and/or lower internal waste mass temperatures in order that hydrogen producing bacteria and/or spores are isolated and active.

This expression is also drawn to describe heat-shock techniques necessary to deactivate certain methanogenic or other non-hydrogen producing bacteria and/or spores, and to activate and isolate hydrogen producing and/or spore producing bacteria, including by pre-treatment or baking of inoculating substrates or materials, or by the temporary and/or permanent increase of temperatures within the landfill, waste mass, or vessel, to levels conducive to the activation, isolation, cultivation, and sustainability of hydrogen producing bacteria and/or spores. This expression is also drawn to an internal landfill, waste mass, or vessel operating temperature range of between 165° and 190° Fahrenheit. This is a temperature range that is conducive to the activation, isolation, cultivation, and sustainability of hydrogen producing bacteria and/or spores. This is a temperature range that typically results in deactivation of methane producing bacteria and/or hydrogen consuming bacteria, and has an upper bound temperature that is below typical solid wastes combustion temperature.

A fifth expression of the invention is drawn upon utilizing various methods for producing and sustaining an acidic condition within the landfill, waste mass, or vessel that is optimum for the activation, isolation, cultivation, and sustainability of hydrogen producing bacteria and/or spores. This may be accomplished by the uniform reintroduction of certain liquids, leachate, gas condensate, or other materials, elements, or liquids as necessary, through various appropriately spaced vertical or horizontal supply and distribution piping, and/or introducing directly as the waste is placed by spraying and/or placing on the surface of the waste (landfill) or prior to placement in a closed vessel. This expression is also drawn to the desired internal landfill, waste mass, or vessel pH operating range of between approximately 3.5 and approximately 8.5 with an acidic, optimal target range of approximately 3.5 to approximately 6.5. This pH range is maintained to provide the best conditions for hydrogen producing bacteria during the fermentation of substrates/waste mass and to exclude/prohibit methanogens or other hydrogen consuming processes.

This expression of the invention is also drawn upon placing certain wastes, or specific other materials containing iron, phosphorous, etc., which purpose is the maintenance of an acidic level conducive to the activation and sustainability of hydrogen producing bacteria and/or spores. This may also be accomplished by the uniform reintroduction of certain liquids, leachate, gas condensate, or other materials or elements, as necessary, through various supply and distribution piping, and/or introducing directly as the waste is placed by spraying and/or placing on the surface of the waste. The injection, introduction, or addition of liquid wastes, certain materials, other liquids, or gaseous amendments, as necessary, is to sustain a specific acidic condition and to retain an acidic phase of fermentation.

A sixth expression of the invention is drawn upon the reduction or elimination of methane gas and methane gas emissions that occurs as a result of typical, present-day landfill operations. Design, operation, and management of a landfill or vessel in accordance with the techniques prescribed herein, results in the production of hydrogen gas in lieu of methane gas, eliminating methane-associated greenhouse gas emissions and atmosphere impacts.

The overall effect of the incorporation of each of the features and management techniques described is the development or conversion of a specific landfill, waste mass, or vessel environment that is conducive to the activation, isolation, cultivation, and sustainability of hydrogen gas producing bacteria and/or spores. This method of managing solid waste provides for the production of gas which contains as one of its primary components free hydrogen gas, which after appropriate clean-up or hydrogen separation processes, produces free hydrogen gas which may be used as a fuel for producing energy directly, as a fuel for hydrogen fuel cells, or for any identified or as yet unidentified, beneficial reuse. This process also has the advantage of significantly reducing greenhouse gas emissions when compared to today's landfill operating techniques.

Referring to the various figures, and in accordance with a preferred embodiment, the preceding goals are achieved by the provision of a landfill facility 10 as described herein. Although a landfill facility 10 is disclosed in accordance with a preferred embodiment of the present invention, the concepts underlying the present invention may be employed in various waste management facility environments without departing from the spirit of the present invention. The present landfill facility 10 includes a variety of design features intended to provide for operational control of the waste mass 12 for ultimate hydrogen production. Certain design features, facilities, and operational techniques are incorporated into the landfill facility 10 or vessel construction (for new construction and/or for conversion of existing facilities as applicable). These are detailed in the following description.

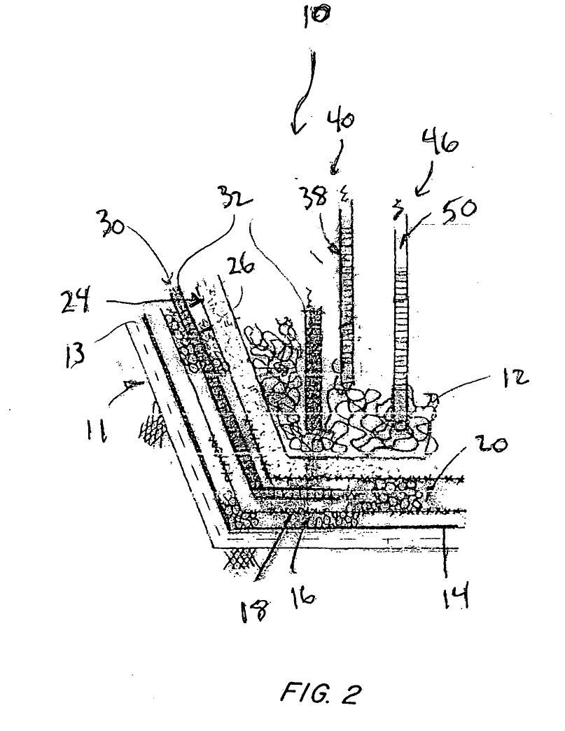

Referring to FIG. 2, the landfill facility 10 includes design and construction used in a typical landfill base grade 11, consistent with all regulations for siting and development. The landfill facility 10 is constructed with a landfill subdrain 13 and/or prescriptive landfill bottom liner system 14 consistent with all regulations and standards. The bottom liner system 14 preferably includes a prescriptive liner section composed high density polyethylene (HDPE) over clay or geocomposite.

The landfill facility 10 includes a landfill leachate collection system, including a leachate collection layer, or drainage layer, 16 and appurtenant piping (not shown), consistent with all regulations and standards. The landfill facility 10 also includes an appropriate geosynthetic layer 18 of geosynthetic materials above the leachate collection layer 16, to separate the leachate collection layer 16 from the next vertical surface layer and a permeable material (sand, small graded rock, etc.) above the landfill bottom liner system 14 and leachate collection layer 16, over the entire landfill bottom surface and sides, as an air injection and circulation layer 20. As will be discussed below in greater detail, air is pumped into the air injection and circulation layer via a landfill air circulation control system 30 including piping 32.

More particularly, the landfill facility 10 includes piping/perforated piping 32 within the air injection and circulation layer 20, at effective air distribution intervals, as discussed below in greater detail, for the introduction of air for producing aerobic conditions and achieving desired waste mass heat levels in the manner discussed below in greater detail. A geosynthetic materials layer 24 is placed above the air injection and circulation layer 20, followed by placement of select fill soil materials or other approved select fill materials for bottom layer(s) protection and for heat insulation purposes, prior to placement of waste materials 12.

In accordance with a preferred embodiment of the present invention, solid waste placement or loading (in a landfill, vessel, or any waste management unit) is conducted to provide for the greatest degree of mixing and homogeneity of all waste placed. Specifically, it is desired to expose as much of the solid waste to specific bacteria containing waste materials, cover material/soils, or other inoculants during the placement process to obtain the best mixing and contact of all wastes. It is also desired to expose the waste mass 12 to waste and/or cover materials that contain hydrogen producing bacteria, and to achieve the incorporation of trace element materials (especially iron; nickel, etc) into the waste mass 12 necessary for optimum hydrogen production.

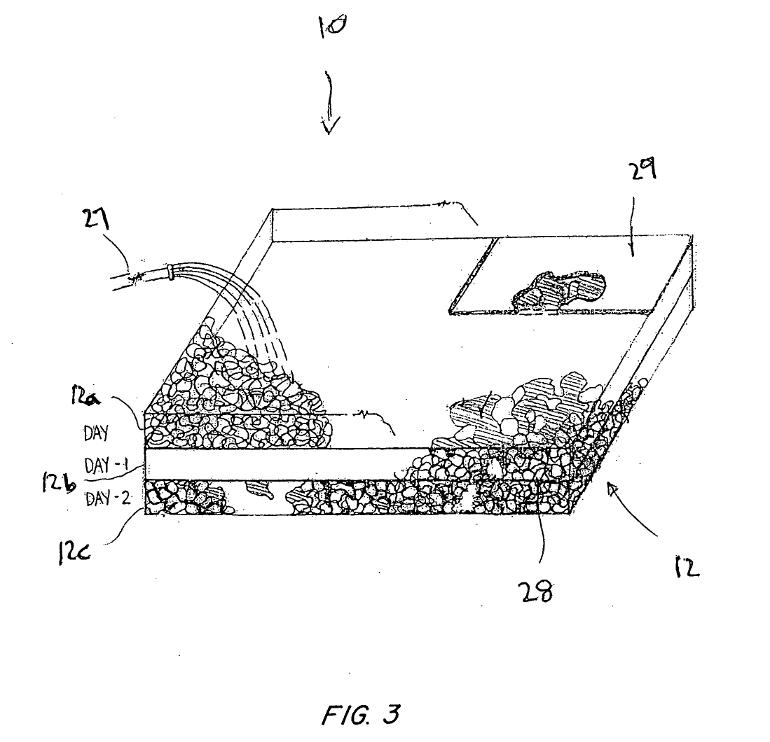

In practice, and referring to FIG. 3, typical solid wastes are placed and spread to relatively thin layers 12a, 12b, 12c of approximately 2-3 feet over the course of an operating day, or operating interval. Each solid waste layer placement is followed by the placement and inoculation of that layer of waste with municipal sludge, selected soils, specific food wastes, any appropriate organic substrate, inoculating materials, soils containing appropriate trace elements (especially iron, nickel, etc.), selective trace element additives, and/or cover soils (see reference numeral 28 which generally represents these various materials) by spreading those materials over/into the thin waste layer. The placement/compaction of the waste is directed to mixing and for obtaining homogeneity of the waste mass, where obtaining maximum density is not necessarily desired nor required. In addition to the direct intermixing of these materials 28 within the waste mass 12, these materials 28 may be added by spraying 27 them onto the waste mass 12 or utilizing a supplemental daily cover tarp, retractable cover and/or other supplemental cover including supplemental inoculating materials 29 to cover the waste mass 12. Uniformity and homogeneity of the waste mass 12, especially with respect to inoculating materials and trace elements, is desired, and is the goal of waste loading (vessel) methods or placement methods (landfill).

As an example, smaller landfill facilities (say less than 1000 tons per day) may place all solid waste received in one, single, two-foot layer (over approximately ½ acre) with other selected wastes (like municipal sludge) being worked in over that layer at the close of the operating day. Alternatively, or in combination with the addition of selected wastes or other materials, cover soils, or soils with naturally occurring hydrogen-producing bacteria, may be placed over the top of the waste layer. It is not necessarily desired to fully cover the waste with soils or selected wastes, but to place specific bacteria containing wastes or soils in order that they work into the waste to uniformly fill voids and to act as inoculants and/or as materials for the supply of necessary trace elements.

Larger landfill facilities may operate in a similar manner building the described, desired two-foot layers of solid waste and specific waste type and or soil placement, and then continuing with similar layer construction above the previously placed layer(s), to any height that may be practical given that facilities daily/weekly capacity.

For vessel controlled operations, waste and other material placement may take place following the general description and recipe for obtaining a homogenous, well mixed solid waste mass. The waste mass may include uniformly distributed inoculants, specific bacteria containing soils, required trace element(s), liquid wastes or other materials selected or prepared specifically for inoculation and sustainability of the waste mass for optimum hydrogen production.

The landfill facility 10 is further provided with a landfill air circulation control facility 30. As waste 12 and inoculating materials 28 are placed within the landfill cell or operating area, and the landfill facility 10 continues to grow in volume and height, additional waste mass landfill air circulation control facilities are placed or extended to enable waste mass environment control. Within a closed vessel these features would be designed into the vessel and incorporated during the vessel construction.

More particularly, and with reference to FIG. 1, at specific vertical and horizontal intervals of landfill development within the landfill facility 10 (at approximate 50 foot vertical intervals from the bottom up, and at approximate 150-200 foot horizontal intervals), piping 32 is installed within permeable trenches or continuous permeable layers, within/across the waste mass connected to, and continuous with, a primary air circulation system (for example, a blower) 34. It is contemplated the blower may be reversed and utilized for venting purposes within the spirit of the present invention. Valves 36 are also installed on all piping 32 at all points of connection to afford air circulation control in specific landfill areas. Adequate landfill side-slope and top cover (soil or other relatively impermeable material) is maintained to prevent air/oxygen intrusion and to provide the ability to control waste mass air/oxygen circulation by controlled means only.

At approximately the same vertical and horizontal intervals used in the placement of piping 32 for the landfill air circulation facility 30, piping 38 is installed within permeable trenches or continuous permeable layers for a liquid recharge or injection (or liquid introduction) system 40. Waste filling is continued as prescribed using the air circulation piping 32 and liquid injection piping 38 at effective intervals, until the filling of the landfill facility 10 is complete. Although the liquid introduction system 40 is shown along the left side of the landfill facility shown in FIG. 1, piping 38 would be found throughout the landfill facility 10 as noted above.

The landfill facility 10 also includes monitoring and control facilities 42 for ensuring optimum operation of the landfill facility 10. Following landfill development by the methods prescribed above, and after a landfill cell or operating area has been completed with fill by the methods described, the monitoring and control facilities 42 are installed. The monitoring and control facilities 42 are used to monitor temperature, waste mass moisture content, waste mass pH, internal waste mass partial gas pressure, and any other physical conditions within the landfill waste mass. In addition, the monitoring and control facilities 42 may be used as points of inoculation or injection, for liquids, substrates, or other additives for achieving desired conditions, including specific internal waste mass partial gas pressures, and for overall control of the waste mass environment.

In accordance with a preferred embodiment the monitoring and control facilities 42 of the landfill facility 10 are achieved by using common landfill vertical drilling techniques, installing vertical column wells, incorporating vertical piping/perforated piping and surrounding sandy/permeable materials as backfill, from the surface of the landfill to the vicinity of the bottom of the landfill. Such techniques are well known within the art, and one implementing the present invention would readily appreciate the various techniques which may be used for the monitoring and control facilities based upon the specific requirements of the landfill implementing the concepts of the present invention. In accordance with a preferred embodiment, the monitoring and control facilities 42 include two (or more) wells (nested wells) 44a, 44b installed at predetermined locations, spaced evenly within the landfill facility in order to monitor the entire landfill facility. The wells 44a, 44b provide for monitoring and control capability over the entire landfill facility 10, generally no more than 200 feet apart (100-foot radius of influence or monitoring capability), or as appropriate as may be dictated by waste mass internal partial gas pressure. One of the wells (nested) 44a may be used for water, liquid, or substrate injection for moisture control or pH control, and may also be used for temperature monitoring. The other well (s) 44b may to be used for temperature monitoring and/or partial gas pressure and ultimately may be used for incorporation into an integrated gas collection system 46, after achieving appropriate hydrogen producing waste mass conditions. Only a first series of these nested wells are shown in FIG. 1 along the upper right of the landfill facility, although those skilled in the art will appreciate additional nested wells would be placed at appropriate locations throughout the landfill facility 10.

The landfill facility 10 also includes an air management system 48 for waste mass temperature control. The air management system 48 ultimately controls operation of the air circulation control facility 30 as described below. Actuation of the air circulation control facility 30 under the control of the air management system 48 is ultimately achieved by linking the monitoring and control facilities 42 thereto for the transfer of information necessary to make operating decisions. Following landfill development, and installation monitoring and control facilities 42 by the methods described above, operations are conducted to obtain temperatures within the entire waste mass 12 (landfill, vessel, etc.) that deactivate methanogenic bacteria, and that are conducive to the isolation, cultivation and sustainability of hydrogen producing bacteria and/or spores. Prior to this step, and while the landfill facility 10 is being developed, typical landfill operational methods are employed to maintain anaerobic conditions.

After the landfill facility unit development is complete, and monitoring and control facilities 42 are installed, operations begin for the ultimate production of hydrogen. This is accomplished by first developing aerobic conditions within the entire waste mass to temporarily (or permanently) elevate the entire waste mass 12 internal temperature to greater than 165° Fahrenheit by controlling the air flow via the air management system 48 operating the air circulation control facility 30 for providing air flow in the waste mass. In particular, through the utilization of the landfill air circulation control facility 30 oxygen content within the waste mass 12 is controlled to optimize aerobic activity. Since the aerobic activity of bacteria within the waste mass 12 produces more heat than anaerobic activity within the waste mass 12, the temperature within the waste mass 12 can be increased or decreased based upon the amount oxygen which is pumped into the waste mass via the landfill air circulation control facility 30 under the control of the air management system 48. In addition to the aerobic bacteria producing more heat, high temperature survivor bacteria also commonly produce more heat than traditional anaerobic bacteria. Once this temperature is achieved consistently throughout the waste mass 12, air circulation is terminated, and anaerobic conditions are returned. The high temperatures deactivate methanogenic bacteria and once anaerobic conditions are restored, provide an environment conducive to the isolation, cultivation and sustainability of anaerobic hydrogen producing bacteria. By implementing the present system, methane production from the waste mass is reduced resulting in a definitive reduction in the production of greenhouse gases through the operation of a waste management facility in accordance with the present invention.

In accordance with a preferred embodiment, air circulation is facilitated by the piping 32 installed in the bottom and side layers, and internal horizontal trenches, by venting or by a forced air system such as the blower of the primary circulation system 34. This is accomplished by opening various valves 36 installed throughout the interconnected air circulation piping of the landfill air circulation control facility 30 or by forcing air through the same system of piping. Aerobic bacterial decomposition is allowed to begin and to continue. Temperatures are monitored via the monitoring and control facilities 42. Air circulation is adjusted, as necessary, in various areas of the landfill facility 10, to promote further aerobic activity, or reduce as necessary where temperatures become elevated (greater than 190° Fahrenheit). Once temperatures in excess of approximately 165° Fahrenheit (minimum) are obtained throughout the waste mass 12, air circulation is ceased by closing all vents 36 and discontinuing forced air pumping and/or passive venting. The landfill waste mass 12 (or vessel) is then operated in an anaerobic condition, keeping air from intrusion into the sides or top of the landfill facility 10, with appropriate cover soil materials or synthetic cover.

Waste mass management for maintaining desired pH and moisture levels is achieved in the following manner. Hydrogen producing bacteria thrive and produce the largest volume percentage of hydrogen gas in a pH environment between approximately 3.5 and approximately 8.5, more preferably, between approximately 3.5 to approximately 6.5. Maintenance and sustainability of the pH environment is accomplished by the placement (landfill), loading (vessel), or intermittent injection or addition of certain liquid wastes, solid wastes, organic wastes, inorganic wastes, trace elements, or other products as necessary to sustain the pH range that is optimum for hydrogen producing bacteria and for hydrogen production. Further, moisture conditions are maintained to provide an optimum level of water availability to promote aerobic and anaerobic decomposition, and in certain instances, may be used to control temperatures. The pH levels are monitored by obtaining gas condensate samples from within monitoring ports/wells 44a, 44b, and/or by sample of leachate generated. The pH levels are adjusted as necessary, for maximum hydrogen production, by the addition of acidic or basic liquids to the liquid introduction system 40. Moisture levels are monitored within the waste mass 12 and adjusted as necessary to produce optimum conditions, by the injection of water or other liquids through the piping 38 of the liquid introduction system 40.

Hydrogen gas is extracted via a gas collection system 46 from the landfill facility 10 (or vessel) for beneficial reuse. The landfill gas collection system 46 is comprised of vertical and/or horizontal collection wells 50, and collection and delivery piping 51 are common within the waste management industry. Although the wells 50 are only shown along the left side of the landfill facility shown in FIG. 1, wells 50 with associated collection and delivery 51 would be positioned throughout the landfill facility. The systems commonly include a vacuum station 54 (commonly referred to as a “blower” unit) connected to an integrated system of wells 50, lateral collection piping 51, and a main pipe (header) (not shown) for delivery to a flare for incineration or to turbine engines for utilization of methane as a fuel for generating electricity.

A collection system common to today's technology would be used to collect hydrogen gas from the landfill (or modified appropriately for a vessel) for delivery to a processing station for clean-up and for ultimate reuse of the free hydrogen component of the gas. Where current state systems are generally designed for the active collection of methane gas, and where the goal of methane gas well-field operations is generally to prevent positive pressure build-up within the landfill to prevent surface emissions, operation of the collection system for hydrogen gas may include more closely spaced wells, additional monitoring and control equipment, and/or various automated systems for sustaining an internal waste mass hydrogen gas partial pressure necessary for the continuous, optimal production of hydrogen gas.

In practice, and in accordance with a preferred embodiment of the present invention, all wells (and/or use existing monitoring ports), lateral piping, and main header piping, as is common for landfill gas extraction, are installed within the landfill facility. Well spacing, especially with respect to gas extraction, is dictated by the need to maintain certain partial gas pressures within in as much of the waste mass as practical, to maintain an internal waste mass hydrogen gas partial pressure necessary for optimal hydrogen production. The landfill gas is collected to a central station 52 by means of a common vacuum unit 54. Free hydrogen gas is extracted from the total gas volume collected by means of existing available, or as yet developed, gas separation or clean-up techniques. Free hydrogen gas is obtained as a target product for commercial, industrial, or other beneficial reuse.

While the preferred embodiments have been shown and described, it will be understood that there is no intent to limit the invention by such disclosure, but rather, is intended to cover all modifications and alternate constructions falling within the spirit and scope of the invention.

1. A waste management facility provided with operational control of a waste mass for optimum hydrogen production, comprising:
an air circulation control facility cooperating with the waste mass;
a liquid introduction system cooperating within the waste mass;
monitoring means for monitoring at least temperature, waste mass moisture content and waste mass pH;
control means for adjusting air circulation and liquid introduction thereby controlling waste mass temperature and pH to create an optimum environment to deactivate methanogenic bacteria and isolate and maximize the number of hydrogen producing bacteria; and
a gas collection system for collecting the hydrogen produced by the hydrogen producing bacteria.

2. The waste management facility according to claim 1, further including a plurality of layers including a bottom liner system; a leachate collection system; a geosynthetic layer above the leachate collection system to separate the leachate collection system from a next vertical surface layer; a permeable material above the bottom liner system and leachate collection system, which functions as an air injection and circulation layer for the introduction of air for producing aerobic conditions and achieving desired waste mass heat levels; a geosynthetic materials layer placed above the air injection and circulation layer; and followed by placement of select fill soil materials.

3. The waste management facility according to claim 2, wherein the leachate collection system includes a drainage layer.

4. The waste management facility according to claim 1, wherein the air circulation control facility includes piping connected to a primary air circulation system.

5. The waste management facility according to claim 4, wherein the air circulation control facility also includes valves installed on the piping to afford air circulation control in specific areas.

6. The waste management facility according to claim 1, wherein solid waste is exposed to hydrogen producing bacteria present in waste materials, cover material/soils, or other inoculants during a placement process to obtain mixing and contact of all wastes.

7. The waste management facility according to claim 1, wherein trace element materials are incorporated into the waste mass for optimum hydrogen production.

8. The waste management facility according to claim 1, wherein inoculating materials are incorporated into the waste mass.

9. The waste management facility according to claim 1, wherein municipal sludge, selected soils, specific food wastes, organic substrates, inoculating materials, soils containing appropriate trace elements, selective trace element additives, or cover soils are incorporated into the waste mass.

10. The waste management facility according to claim 1, wherein the monitoring means and the control means are part of a monitoring and control facilities.

11. The waste management facility according to claim 10, wherein the monitoring and control facilities are used as a point of inoculation or injection for liquids, substrates, or other additives for achieving desired conditions.

12. The waste management facility according to claim 1, wherein the monitoring and control facilities include first and second wells, wherein the first well is used for water, liquid, or substrate injection for moisture or pH control or for temperature monitoring and the second well is used for temperature monitoring, partial gas pressure, or for incorporation into an integrated gas collection system.

13. The waste management facility according to claim 1, wherein the waste mass is elevated to an internal temperature greater than 165° Fahrenheit by controlling the air flow in the waste mass in a manner deactivating methanogenic bacteria.

14. The waste management facility according to claim 13, wherein the temperature of the waste mass does not exceed approximately 190° Fahrenheit.

15. The waste management facility according to claim 14, wherein the waste mass has a pH between approximately 3.5 to approximately 6.5.

16. The waste management facility according to claim 15, wherein acidic or basic liquids are added to the waste mass via the liquid introduction system

17. The waste management facility according to claim 1, wherein the waste mass has a pH between approximately 3.5 to approximately 6.5.

18. The waste management facility according to claim 17, wherein acidic or basic liquids are added to the waste mass via the liquid introduction system.

19. The waste management facility according to claim 1, wherein the waste management facility is a landfill facility.

20. The waste management facility according to claim 1, wherein a reduction in the production of greenhouse gases is achieved.
